(19) United States
(12) Patent Application Publication
(10) Pub. No.: US 2014/0378409 A1
(43) Pub. Date: Dec. 25, 2014

(54) EFFECT POTENTIATOR FOR ANTITUMOR AGENTS

(71) Applicant: TAIHO PHARMACEUTICAL CO., LTD., Chiyoda-ku, Tokyo (JP)

(72) Inventors: Hidenori Fujita, Tsukuba-shi (JP); Masanori Kato, Tsukuba-shi (JP)

(73) Assignee: TAIHO PHARMACEUTICAL CO., LTD., Chiyoda-ku, Tokyo (JP)

(21) Appl. No.: 14/369,060

(22) PCT Filed: Dec. 27, 2012
(86) PCT No.: PCT/JP2012/083794

§ 371 Date: Jun. 26, 2014

(30) Foreign Application Priority Data
Dec. 28, 2011 (JP) 2011-290125

(51) Int. Cl. A61K 31/47; A61K 45/06; A61K 31/4745; A61K 31/513; A61K 31/517; A61K 31/337; A61K 31/7068

(52) U.S. Cl. CPC A61K 31/47; A61K 31/337; A61K 45/06; A61K 31/7068; A61K 31/513; A61K 31/517; A61K 31/4745
USPC 514/49; 514/312; 514/274; 514/266.24; 514/283

(57) ABSTRACT

An antitumor effect potentiator of another antitumor agent, including an acylthiourea compound represented by formula (I) or a pharmaceutically acceptable salt thereof as an active ingredient:

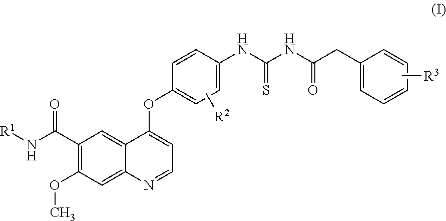

wherein R¹ represents a C₁₋₆ alkyl group which may have a substituent, while the substituent represents any one of a hydroxyl group, a C₃₋₁₀ cycloalkyl group, a C₁₋₆ alkoxy group which may have a substituent, a C₁₋₆ alkylamino group which may have a substituent, a C₁₋₆ alkanoylamino group, a C₁₋₆ alkylsulfonyl group, a C₆₋₁₄ aromatic hydrocarbon group which may have a substituent, a saturated or unsaturated heterocyclic group which may have a substituent, a C₁₋₆ alkylaminocarbonyl group which may have a substituent, a saturated or unsaturated heterocyclic carbonyl group which may have a substituent; R² represents a fluorine atom or a chlorine atom; and R³ represents a hydrogen atom, a fluorine atom, or a chlorine atom.

### FIELD OF THE INVENTION

The present invention relates to an agent for potentiating antitumor effect and an antitumor drug comprising a combination of another antitumor agent.

### BACKGROUND OF THE INVENTION

There are various kinds of antitumor agents, and are broadly classified into, for example, alkylating agents, platinum compounds, antimetabolite agents, topoisomerase inhibitors, microtubule inhibitors, antitumor antibiotics, molecular target drugs. Also, in recent years, antitumor agents are frequently used in combination therapy, rather than administered alone.

For example, combination therapy of 4-(3-chloro-4-(cyclopropylaminocarbonyl)aminophenoxy)-7-methoxy-6-quinolinecarboxamide, which is an angiogenesis inhibitor having a VEGF receptor kinase inhibitory activity, with Taxane has been reported (Patent Document 1).

On the other hand, c-Met is a receptor tyrosine kinase which is classified as one of proto-oncogenes. It has been reported that, when c-Met is overexpressed, mutated or translocated frequently in various kinds of cancers (for example, renal cell cancer, gastric cancer, lung cancer, colorectal cancer, pancreatic cancer, ovarian cancer, hepatic cell cancer, head and neck cancer, melanoma), activation of c-Met is enhanced, and leads to cell proliferation, infiltration/metastasis, tumor formation, neovascularization or anti-apoptosis (for example, Non-Patent Document 1). Therefore, a compound having a c-Met inhibiting effect is known as a useful antitumor agent (Patent Document 2). Combination of a quinoline derivative which is a c-Met inhibitor and is a multikinase inhibitor including c-Met and AXL, with an ErbB inhibitor, has also been reported (Patent Document 3).

### CITATION LIST

### Patent Documents

Patent Document 1: WO 2009/096377
Patent Document 2: WO 2009/125597
Patent Document 3: WO 2009/137429
Non-Patent Document 1: J. Cell Biol. 111, p. 2097-2108 (1990)

### SUMMARY OF THE INVENTION

### Problem to be Solved by the Invention

However, it is unknown whether, when a certain antitumor agent and another antitumor agent are used in combination, their antitumor effect is potentiated, or the effect is weakened.

An object of the present invention is to provide a combination of two kinds of antitumor agents used in combination, by which their antitumor effect is potentiated.

### Means for Solving the Problem

Thus, the inventors of the present invention has selected a certain kind of c-Met inhibitor, and studied the activity in the case of using the compound and another antitumor agent in combination. The inventors have found that an acylthiourea compound having an aminocarbonyl group as a substituent at 6-position and having an alkoxy group as a substituent at 7-position of a quinoline ring represented by the following formula (I), or a pharmaceutically acceptable salt thereof, shows strong c-Met inhibiting activity with reduced adverse side effects. And the inventors also have found that when used in combination with another antitumor agent, the acylthiourea compound or a salt thereof extends the efficacy range or the antitumor spectrum by an excellent antitumor effect potentiating activity, thus completed the present invention.

The present invention relates to the following [1] to [8].

[1] An agent for potentiating antitumor effect of another antitumor agent, comprising an acylthiourea compound represented by the following formula (I):

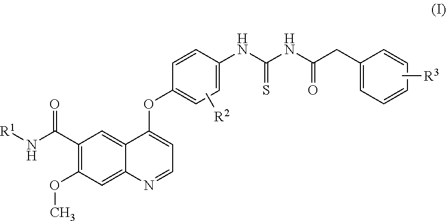

wherein R¹ represents a C₁₋₆ alkyl group which may have a substituent, while the substituent represents any one of a hydroxyl group, a C₃₋₁₀ cycloalkyl group, a C₁₋₆ alkoxy group which may have a substituent, a C₁₋₆ alkylamino group which may have a substituent, a C₁₋₆ alkanoylamino group, a C₁₋₆ alkylsulfonyl group, a C₆₋₁₄ aromatic hydrocarbon group which may have a substituent, a saturated or unsaturated heterocyclic group which may have a substituent, a C₁₋₆ alkylaminocarbonyl group which may have a substituent, a saturated or unsaturated heterocyclic carbonyl group which may have a substituent;

R² represents a fluorine atom or a chlorine atom; and

R³ represents a hydrogen atom, a fluorine atom, or a chlorine atom.

[2] An antitumor drug comprising a combination of the acylthiourea compound represented by formula (I) or a pharmaceutically acceptable salt thereof, with another antitumor agent.

[3] An acylthiourea compound represented by formula (I) or a pharmaceutically acceptable salt thereof, for potentiating the antitumor effect of another antitumor agent.

[4] An acylthiourea compound represented by formula (I) or a pharmaceutically acceptable salt thereof, for treating a tumor by administering the compound or the salt together with another antitumor agent as a single dosage or as a separate dosage.

[5] Use of an acylthiourea compound represented by formula (I) or a pharmaceutically acceptable salt thereof, for the manufacture of an antitumor effect potentiator for another antitumor agent.

[6] Use of an acylthiourea compound represented by formula (I) or a pharmaceutically acceptable salt thereof, for the manufacture of an antitumor drug comprising a combination of the compound and another antitumor agent.

[7] A method for potentiating an antitumor effect of another antitumor agent, the method including administering an acylthiourea compound represented by formula (I) or a pharmaceutically acceptable salt thereof and the antitumor agent to a patient in need thereof.

[8] A method for treating a tumor, the method including administering an acylthiourea compound represented by formula (I) or a pharmaceutically acceptable salt thereof and another antitumor agent to a patient in need thereof.

### Effects of the Invention

The acylthiourea compound (I) or a pharmaceutically acceptable salt thereof potentiates the effect of various antitumor agents when used in combination with these antitumor agents. Specifically, 1) because the acylthiourea compound (I) does not potentiate most of the adverse side effects of individual agents of the antitumor agents used in combination, the compound can be used in combination at the respective maximum effect-exhibiting doses of the respective single agents, without reducing the effective doses of the antitumor agents used in combination; 2) the compound potentiates the antitumor effect of the agents used in combination, irrespective of the drug sensitivity of the antitumor agents used in combination; and 3) the antitumor effect is observed even at a low dose at which the acylthiourea compound (I) itself does not exhibit any antitumor effect. As a result, the present invention provides a therapeutic method with high clinical usefulness, such as extension of the efficacy range for cancer treatment and enhancement of therapeutic effects.

### DESCRIPTION OF DRAWINGS

FIG. 1 illustrates a combined effect of 200 mg/kg/day of compound (1) and paclitaxel on human lung cancer cell line NCI-H460 subcutaneously transplanted model (nude mice).

FIG. 2 illustrates the body weight change in the case of combined use of 200 mg/kg/day of compound (1) and paclitaxel on human lung cancer cell line NCI-H460 subcutaneously transplanted model (nude mice).

FIG. 3 illustrates a combined effect of 200 mg/kg/day of compound (1) and gemcitabine on human lung cancer cell line NCI-H441 subcutaneously transplanted model (nude mice).

FIG. 4 illustrates a combined effect of 50 mg/kg/day and 250 mg/kg/day of compound (1) and TS-1 on human gastric cancer cell line NUGC-4 subcutaneously transplanted model (nude rats).

FIG. 5 illustrates a combined effect of 200 mg/kg/day of compound (1) and lapatinib on human gastric cancer cell line NCI-N87 subcutaneously transplanted model (nude mice).

FIG. 6 illustrates a combined effect of 50 mg/kg/day of compound (1) and irinotecan hydrochloride on human colorectal cancer cell line HT-29 subcutaneously transplanted model (nude mice).

FIG. 7 illustrates the body weight change in the case of combined use of 50 mg/kg/day of compound (1) and irinotecan hydrochloride on human colorectal cancer cell line HT-29 subcutaneously transplanted model (nude mice).

FIG. 8 illustrates a combined effect of 12.5 mg/kg/day of compound (1) and paclitaxel on human gastric cancer cell line NUGC-4 subcutaneously transplanted model (nude mice).

FIG. 9 illustrates the body weight change in the case of combined use of 12.5 mg/kg/day of compound (1) and paclitaxel on human gastric cancer cell line NUGC-4 subcutaneously transplanted model (nude mice).

### DETAILED DESCRIPTION OF THE INVENTION

In regard to the acylthiourea compound (I) of the present invention or a pharmaceutically acceptable salt thereof, the phrase “which may have a substituent” or “optionally substituted” used for a certain structure means that the structure may have one or two or more “substituents” at a chemically acceptable position(s) on the structure.

The kind of the substituent present in the structure, the number of substituents, and the position of substitution are not particularly limited, and when two or more substituents are present, they may be the same or different from each other. Examples of the “substituent” include a halogen atom, a hydroxyl group, a cyano group, a nitro group, a C₁₋₆ alkanoyl group, a C₁₋₆ alkyl group, a C₃₋₁₀ cycloalkyl group, a C₂₋₆ alkenyl group, a C₁₋₆ alkoxy group, an amino group, a C₁₋₆ alkylamino group, a C₁₋₆ alkanoylamino group, a C₁₋₆ alkylaminocarbonyl group, a C₁₋₆ alkylsulfonyl group, a C₆₋₁₄ aromatic hydrocarbon group, a saturated or unsaturated heterocyclic group, a saturated or unsaturated heterocyclic carbonyl group, and an oxo group. When the above substituents are present, the number is typically 1 to 3.

In the formula (I), the “C₁₋₆ alkyl group” of the “C₁₋₆ alkyl group which may have a substituent” represented by R¹ represents a linear or branched alkyl group having 1 to 6 carbon atoms, and examples thereof include a methyl group, an ethyl group, an n-propyl group, an isopropyl group, an n-butyl group, a sec-butyl group, a tert-butyl group, an n-pentyl group, an isopentyl group, an n-hexyl group, and an isohexyl group. A C₁₋₄ alkyl group is more preferred, and a methyl group, an ethyl group, an n-propyl group, an isopropyl group, an n-butyl group, and a sec-butyl group are particularly preferred.

In the formula (I), The “C₃₋₁₀ cycloalkyl group” indicated as a substituent of the “C₁₋₆ alkyl group which may have a substituent” of R¹ represents a cycloalkyl group having 3 to 10 carbon atoms, and examples thereof include a cyclopropyl group, a cyclobutyl group, a cyclopentyl group, and a cyclohexyl group, while a cyclohexyl group is more preferred.

In the formula (I), the “C₁₋₆ alkoxy group” of the “C₁₋₆ alkoxy group which may have a substituent” indicated as a substituent of the “C₁₋₆ alkyl group which may have a substituent” of R¹ represents a linear or branched alkoxy group having 1 to 6 carbon atoms, and examples thereof include a methoxy group, an ethoxy group, an n-propyloxy group, an isopropyloxy group, an n-butyloxy group, a sec-butyloxy group, a tert-butyloxy group, an n-pentyloxy group, and an n-hexyloxy group, while a methoxy group, an ethoxy group, and an isopropyloxy group are particularly preferred.

In the formula (I), the “substituent” of the “C₁₋₆ alkoxy group which may have a substituent” indicated as a substituent of the “C₁₋₆ alkyl group which may have a substituent” of R¹ is preferably a hydroxyl group.

In the formula (I), the “C₁₋₆ alkylamino group” of the “C₁₋₆ alkylamino group which may have a substituent” indicated as a substituent of the “C₁₋₆ alkyl group which may have a substituent” of R¹ represents an amino group that is mono-substituted or di-substituted with the C₁₋₆ alkyl group described above, and examples thereof include a methylamino group, an ethylamino group, a dimethylamino group, a methylethylamino group, an n-propylamino group, an isopropylamino group, an n-butylamino group, a sec-butylamino group, a tert-butylamino group, an n-pentylamino group, and an n-hexylamino group, while a diethylamino group is more preferred.

In the formula (I), examples of the “C₁₋₆ alkanoylamino group” indicated as a substituent of the “C₁₋₆ alkyl group which may have a substituent” of R¹ include a formyl group, an acetyl group, a propionyl group, and a butyryl group, and an acetylamino group is more preferred.

In the formula (I), the “C₁₋₆ alkylsulfonyl group” indicated as a substituent of the “C₁₋₆ alkyl group which may have a substituent” of R¹ represents a sulfonyl group substituted with the C₁₋₆ alkyl group described above, and a methylsulfonyl group is more preferred.

In the formula (I), the “C₆₋₁₄ aromatic hydrocarbon group” of the “C₆₋₁₄ aromatic hydrocarbon group which may have a substituent” indicated as a substituent of the “C₁₋₆ alkyl group which may have a substituent” of R¹ represents an aromatic hydrocarbon group having 6 to 14 carbon atoms, and examples thereof include a phenyl group and a naphthyl group, while a phenyl group is more preferred.

In the formula (I), the “saturated or unsaturated heterocyclic group” of the “saturated or unsaturated heterocyclic group which may have a substituent” indicated as a substituent of the “C₁₋₆ alkyl group which may have a substituent” of R¹ represents a monocyclic or bicyclic saturated or unsaturated heterocyclic group having any one or two atoms of an oxygen atom, a nitrogen atom and a sulfur atom, and examples thereof include a pyrrolidinyl group, a piperidinyl group, a piperazinyl group, a morpholino group, a thiomorpholino group, a homopiperidinyl group, a tetrahydrothienyl group, an imidazolyl group, a thienyl group, a furyl group, a pyrrolyl group, an oxazolyl group, an isoxazolyl group, a thiazolyl group, an isothiazolyl group, a pyrazolinyl group, a triazolyl group, a tetrazolyl group, a pyridyl group, a pyrazyl group, a pyrimidinyl group, a pyridazyl group, an indolyl group, an isoindolyl group, an indazolyl group, a methylenedioxyphenyl group, an ethylenedioxyphenyl group, a benzofuranyl group, a dihydrobenzofuranyl group, a benzimidazolyl group, a benzoxazole group, a benzothiazolyl group, a purinyl group, a quinolyl group, an isoquinolyl group, a quinazolinyl group, and a quinoxalyl group. A 5-membered to 7-membered heterocyclic group having one to four of nitrogen atoms and/or oxygen atoms is more preferred, and a pyrrolidinyl group, a morpholino group, a dioxolane group, a tetrahydropyranyl group, a pyridyl group, and a tetrazolyl group are particularly preferred. The saturated or unsaturated heterocyclic group may further have a substituent, and the substituent is preferably a C₁₋₆ alkyl group (particularly, a methyl group), or an oxo group.

In the formula (I), the “C₁₋₆ alkylaminocarbonyl group” of the “C₁₋₆ alkylaminocarbonyl group which may have a substituent” indicated as a substituent of the “C₁₋₆ alkyl group which may have a substituent” of R¹ represents a carbonyl group having the C₁₋₆ alkylamino group described above, and an ethylaminocarbonyl group, a dimethylamino group, and a methylbutylamino group are more preferred. The C₁₋₆ alkylaminocarbonyl group may further have a substituent, and the substituent is preferably a hydroxyl group or a C₁₋₆ alkoxy group (particularly, a methoxy group).

In the formula (I), the “saturated or unsaturated heterocyclic group carbonyl group” of the “saturated or unsaturated heterocyclic group carbonyl group which may have a substituent” indicated as a substituent of the “C₁₋₆ alkyl group which may have a substituent” of R¹ represents a carbonyl group having the saturated or unsaturated heterocyclic group described above, and a 5-membered to 7-membered saturated heterocyclic carbonyl group having one to two atoms of nitrogen atoms and/or oxygen atoms is more preferred, while a pyrrolidinylcarbonyl group and a morpholinocarbonyl group are particularly preferred. The saturated or unsaturated heterocyclic carbonyl group may further have a substituent, and the substituent is preferably a halogen atom (particularly, a fluorine atom), or a C₁₋₆ alkyl group (particularly, a methyl group) which may have a hydroxyl group.

Preferred examples of the C₁₋₆ alkyl group of the C₁₋₆ alkyl group which may have a substituent, which is represented by R¹, include a methyl group, an ethyl group, an n-propyl group, an isopropyl group, an n-butyl group and a sec-butyl group, and preferred examples of the substituent on the alkyl group include a hydroxyl group, a cyclohexyl group, a methoxy group, an ethoxy group, an isopropyloxy group, a diethylamino group, an acetylamino group, a methylsulfonyl group, a phenyl group, a pyrrolidinyl group, a morpholino group, a dioxolane group, a tetrahydropyranyl group, a pyridyl group, a triazolyl group, an ethylaminocarbonyl group, a dimethylaminocarbonyl group, a methylbutylaminocarbonyl group, a pyrrolidinylcarbonyl group, and a morpholinocarbonyl group. The alkoxy group may further have a hydroxyl group as a substituent, the heterocyclic group may further have a methyl group or an oxo group as a substituent, the alkylaminocarbonyl group may further have a hydroxyl group or a methoxy group as a substituent, and the heterocyclic carbonyl group may further have a fluorine atom, or a methyl group which may have a hydroxyl group as a substituent.

Preferred examples of the C₁₋₆ alkyl group of the C₁₋₆ alkyl group which may have a substituent, which is represented by R¹, include a methyl group, an ethyl group, an n-propyl group, an isopropyl group, an n-butyl group and a sec-butyl group, and preferred examples of the substituent on the alkyl group include a hydroxyl group, a methoxy group and a morpholino group.

More preferred examples of R¹ include a methyl group, a methoxyethyl group, a morpholinoethyl group, a morpholinocarbonylmethyl group, a 2-hydroxy-n-butyl group, a 2-hydroxy-2-methyl-n-propyl group, and a 1-hydroxy-n-butan-2-yl group, and in the case of a 1-hydroxy-n-butan-2-yl group, an S-form is preferred.

The position of substitution of R² is preferably the 2-position or the 3-position, and particularly preferably the 2-position. Furthermore, R² is preferably a fluorine atom or a chlorine atom, and more preferably a fluorine atom.

The position of substitution of R³ is preferably the 2-position or the 4-position. Furthermore, R¹ is preferably a hydrogen atom, a fluorine atom or a chlorine atom, and more preferably a hydrogen atom or a fluorine atom.

Among the compounds represented by formula (I), a compound in which R¹ represents a methyl group, an ethyl group, an n-propyl group, an isopropyl group, an n-butyl group or a sec-butyl group, all of which may have a substituent; the substituent on the alkyl group is a hydroxyl group, a methoxy group or a morpholino group; R² represents a fluorine atom; and R³ represents a hydrogen atom or a fluorine atom, is preferred.

Furthermore, specific preferred examples of the compound represented by formula (I) include the following compounds.
(1)
4-(2-Fluoro-4-(3-(2-phenylacetyl)thioureido)phenoxy)-7-methoxy-N-m ethylquinoline-6-carboxamide (compound (1))
(2)
4-(2-Fluoro-4-(3-(2-phenylacetyl)thioureido)phenoxy-7-methoxy-N-(2-morpholinoethyl)quinoline-6-carboxamide (compound (2))
(3)
(S)-4-(2-fluoro-4-(3-(2-(4-fluorophenyl)acetyl)thioureido)phenoxy)-N-(1-hydroxybutan-2-yl)-7-methoxyquinoline-6-carboxamide (compound (3))

Examples of the pharmaceutically acceptable salt of the acylthiourea compound (I) include acid addition salts with inorganic acids such as hydrochloric acid, hydrobromic acid, hydroiodic acid, sulfuric acid, nitric acid, and phosphoric acid; acid addition salts with organic acids such as formic acid, acetic acid, propionic acid, oxalic acid, malonic acid, succinic acid, fumaric acid, maleic acid, lactic acid, malic acid, citric acid, tartaric acid, carbonic acid, picric acid, methanesulfonic acid, para-toluenesulfonic acid, and glutamic acid; salts with inorganic bases such as sodium, potassium, magnesium, calcium, and aluminum; salts with organic bases such as methylamine, ethylamine, meglumine, and ethanolamine; salts with basic amino acids such as lysine, arginine, and ornithine; and ammonium salts. Furthermore, the acylthiourea compound (I) also includes enantiomers, and also includes hydrates.

The acylthiourea compound (I) that exhibits an antitumor effect potentiating activity according to the present invention can be produced by the method described in WO 2009/125597.

The acylthiourea compound (I) is an antitumor agent showing an excellent c-Met inhibitory activity and having reduced adverse side effects. However, when used in combination with one of various other antitumor agents (hereinafter, referred to as antitumor agent A), the acylthiourea compound (I) has an activity of potentiating the antitumor effect of the antitumor agent A without exhibiting significant deterioration of toxicity.

Although the antitumor agent A whose activity is potentiated by the acylthiourea compound (I) is not particularly limited, examples thereof include antitumor antibiotic substances such as doxorubicin, doxil, and epirubicin; alkylating agents such as cyclophosphamide and nimustine; platina preparations such as cisplatin, carboplatin, and oxaliplatin; pyrimidine-based antimetabolite agents such as 5-fluorouracil (5-FU), tegafur-gimeracil-oteracil potassium (TS-1, generic name: “tegafur-gimeracil-oteracil potassium combination drug” (trade name: “TS-1”)), tegafur-uracil (UFT, generic name: “tegafur-uracil combination drug” (trade name: “UFT”)), capecitabine, doxifluridine, 5-fluoro-2′-deoxyuridine (FdUrd), gemcitabine, and cytarabine; purine-based antimetabolite agents such as fludarabine, cladribine, and nelarabine; folic acid antimetabolite agents such as pemetrexed and methotrexate; plant alkaloid-based antitumor agents such as paclitaxel (for example, TAXOL, ABRAXANE), docetaxel, and irinotecan; low molecular weight molecular targeted drugs such as gefitinib, erlotinib, lapatinib, everolimus, and temsirolimus; and antibody molecular targeted drugs such as bevacizumab, trastuzumab, cetuximab, and rituximab. Among these, pyrimidine-based antimetabolite agents, folic acid antimetabolite agents, plant alkaloid-based antitumor agents, and low molecular weight molecular targeted drugs are preferred.

Preferred examples of the antitumor agent A whose activity is potentiated by the acylthiourea compound (I) include paclitaxel (for example, TAXOL, ABRAXANE), gemcitabine, lapatinib, a tegafur-gimeracil-oteracil potassium combination drug, and irinotecan.

Although the malignant tumor that can be treated by the acylthiourea compound (I) together with the antitumor agent A whose activity is potentiated is not particularly limited, examples include head and neck cancer, esophageal cancer, gastric cancer, colon cancer, rectal cancer, liver cancer, gall bladder/bile duct cancer, pancreatic cancer, lung cancer, breast cancer, ovarian cancer, cervical cancer, endometrial cancer, renal cancer, urinary bladder cancer, prostate cancer, testicular tumor, bone-soft tissue sarcoma, leukemia, malignant lymphoma, multiple myeloma, skin cancer, and brain tumor.

When the acylthiourea compound (I) or a pharmaceutically acceptable salt thereof is blended with the antitumor agent A, an antitumor drug having a potentiated antitumor effect is obtained. The form of such a new antitumor drug may be a single dosage form containing the acylthiourea compound (I) or a pharmaceutically acceptable salt thereof and the antitumor agent A, or may be a separate dosage form, in which a preparation containing the acylthiourea compound (I) or a pharmaceutically acceptable salt and a preparation containing the antitumor agent A are separate. Furthermore, the means of administration of a composition containing the acylthiourea compound (I) and the means of administration of a composition containing the antitumor agent A may be the same, or may be different (for example, oral administration and injection).

In the case of incorporating the acylthiourea compound (I) or a pharmaceutically acceptable salt thereof into a pharmaceutical composition, the compound is blended with a pharmacological carrier as necessary, and various forms of administration can be employed according to the purpose of prevention or treatment. Examples of the form include an oral preparation, an injectable preparation, a suppository preparation, an ointment preparation, and a patch preparation, however an oral preparation is preferred. These forms of administration can be respectively produced by formulation methods that are commonly known to those ordinarily skilled in the art.

Regarding the pharmacological carrier, various organic or inorganic carrier materials that are commonly used as materials for formulation are used, and the pharmacological carriers are incorporated as an excipient, a binder, a disintegrant, a lubricating agent and a colorant in solid preparations; and as a solvent, for example, a dissolution aid, a suspending agent, an isotonic agent, a buffering agent, a soothing agent in liquid preparations. Furthermore, if necessary, formulation additives such as an antiseptic, an antioxidant, a colorant, a sweetening agent, and a stabilizer can also be used.

When an oral solid preparation is prepared, an excipient, and if necessary, for example, a binder, a disintegrant, a lubricating agent, a colorant, a taste masking or flavoring agent may be added to the compound of the present invention, and then, for example, a tablet, a coated tablet, a granular preparation, a powder preparation, a capsule may be produced by conventional methods.

Examples of the excipient include lactose, sucrose, D-mannitol, glucose, starch, calcium carbonate, kaolin, microcrystalline cellulose, and silicic acid anhydride.

Examples of the binder include water, ethanol, 1-propanol, 2-propanol, simple syrup, a glucose solution, an α-starch solution, a gelatin solution, D-mannitol, carboxymethyl cellulose, hydroxypropyl cellulose, hydroxypropyl starch, methyl cellulose, ethyl cellulose, shellac, calcium phosphate, and polyvinylpyrrolidone.

Examples of the disintegrant include dried starch, sodium alginate, powdered agar, sodium hydrogen carbonate, calcium carbonate, sodium lauryl sulfate, stearic acid monoglyceride, and lactose.

Examples of the lubricating agent include purified talc, sodium stearate, magnesium stearate, borax, and polyethylene glycol.

Examples of the colorant include titanium oxide and iron oxide.

Examples of the taste masking/flavoring agent include glucose, orange peel, citric acid, and tartaric acid.

If necessary, the coating or enteric coating for the purpose of sustaining the effect may be performed according to a known coating method in oral preparations. Examples of such a coating agent include hydroxypropylmethyl cellulose, ethyl cellulose, hydroxymethyl cellulose, hydroxypropyl cellulose, polyoxyethylene glycol, and Tween 80 (registered trademark).

When an oral liquid preparation is prepared, for example, a taste masking agent, a buffering agent, a stabilizer, a flavoring agent are added to the acylthiourea compound (I), and, for example, a liquid preparation for internal use, a syrup preparation, an elixir preparation can be prepared by conventional methods. In this case, examples of the taste masking/flavoring agent include those described above, examples of the buffering agent include sodium citrate, and examples of the stabilizer include tragacanth, gum arabic, and gelatin.

When an injectable preparation is prepared, for example, a pH adjusting agent, a buffering agent, a stabilizer, an isotonic agent, a topical anesthetic agent are added to the acylthiourea compound (I), and subcutaneous, intramuscular and intravenous injectable preparations can be prepared by conventional methods. Examples of the pH adjusting agent and the buffering agent in this case include sodium citrate, sodium acetate and sodium phosphate. Examples of the stabilizer include sodium pyrosulfite, EDTA, thioglycolic aid, and thiolactic acid. Examples of the topical anesthetic agent include procaine hydrochloride and lidocaine hydrochloride. Examples of the isotonic agent include sodium chloride, glucose, D-mannitol, and glycerin.

When a suppository preparation is prepared, a carrier for formulation that is known in the art, for example, polyethylene glycol, lanolin, cacao fat, or fatty acid triglyceride, is added to the compound of the present invention, a surfactant such as, for example, Tween 80 (registered trademark) are further added thereto as necessary, and then a suppository preparation can be prepared by a conventional method.

When an ointment preparation is prepared, for example, a base, a stabilizer, a moisturizing agent, a preservative that are conventionally used are blended with the compound of the present invention as necessary, and an ointment is mixed and formulated by a conventional method. Examples of the base include liquid paraffin, white petrolatum, white beeswax, octyl dodecyl alcohol, and paraffin. Examples of the preservative include methyl para-oxybenzoate, ethyl para-oxybenzoate, and propyl para-oxybenzoate.

When a patch preparation is prepared, for example, the ointment, cream, gel, paste may be applied on a conventional support by a conventional method. Regarding the support, for example, a woven fabric or nonwoven fabric made of cotton, staple fiber, or chemical fiber; or a film or a foam sheet made of soft vinyl chloride, polyethylene, polyurethane is appropriate.

The amount of the acylthiourea compound (I) that should be incorporated in the various unit dosage forms described above may be varied depending on, for example, the symptoms of the patient to which this compound should be applied, or the dosage form. However, generally, the amount per unit dosage form is about 0.05 mg to 2,000 mg in an oral preparation, about 0.01 mg to 100 mg in an injectable preparation, and about 1 mg to 2,000 mg in a suppository preparation.

Furthermore, the amount of administration per day of a medicament having the above-described form of administration varies depending on, for example, the symptoms, body weight, age, gender of the patient, and thus cannot be determined in a generalized manner. However, the amount of administration per day of an adult (body weight: 50 kg) is usually about 0.05 mg to 5,000 mg, and preferably 0.1 mg to 2,000 mg, and it is preferable to administer this amount once a day, or in about two or three divided portions per day.

When a preparation containing the acylthiourea compound (I) or a salt thereof and a preparation containing the antitumor agent A are separate preparations, the respective preparations can be administered simultaneously, or one component can be administered at an arbitrary time point before or after the administration of the other component.

The proportion of administration or mixing of the acylthiourea compound (I) or a pharmaceutically acceptable salt thereof and the antitumor agent A is not particularly limited as long as the proportion is in a range capable of providing an effect of potentiating the antitumor effect, however the proportion of the acylthiourea compound (I) or a pharmaceutically acceptable salt thereof may be about 0.001 moles to 100 moles, and preferably about 0.005 moles to 50 moles, relative to 1 mole of the antitumor agent A.

For example, when the antitumor agent A is paclitaxel (for example, TAXOL, ABRAXANE), the proportion of the acylthiourea compound (I) or a pharmaceutically acceptable salt thereof is about 0.05 moles to 50 moles relative to 1 mole of paclitaxel (for example, TAXOL, ABRAXANE). When the antitumor agent A is gemcitabine, the proportion of the acylthiourea compound (I) or a pharmaceutically acceptable salt thereof is about 0.005 moles to 5 moles relative to 1 mole of gemcitabine. When the antitumor agent A is lapatinib, the proportion of the acylthiourea compound (I) or a pharmaceutically acceptable salt thereof is about 0.01 moles to 20 moles relative to 1 mole of lapatinib. When the antitumor agent A is a tegafur-gimeracil-oteracil potassium combination drug, the proportion of the acylthiourea compound (I) or a pharmaceutically acceptable salt thereof is about 0.05 moles to 50 moles relative to 1 mole of tegafur. When the antitumor agent A is irinotecan, the proportion of the acylthiourea compound (I) or a pharmaceutically acceptable salt thereof is about 0.05 moles to 30 moles relative to 1 mole of irinotecan. When the antitumor agent A is pemetrexed, the proportion of the acylthiourea compound (I) or a pharmaceutically acceptable salt thereof is about 0.05 moles to 20 moles relative to 1 mole of pemetrexed.

### EXAMPLES

Hereinafter, the present invention is described in more detail by way of Examples and Test Examples, however the present invention is not intended to be limited to these Examples. Furthermore, regarding the dose setting of various antitumor agents having their antitumor effects potentiated by the compound of the present invention as illustrated in the following Test Examples, the maximum tolerated dose (MTD) or the maximum dose that can be administered in view of properties, which have been indicated in articles and reports were used.

For antitumor agents, the dose exhibiting the maximum efficacy and the dose exhibiting toxicity are extremely close, and in order to evaluate the maximum antitumor effect of the medicament using an animal model, it is general to evaluate the effect in the vicinity of the MTD. In the following Test Examples, the MTD and the maximum effect-exhibiting dose are considered to have the same meaning.

### Example 1

4-(2-Fluoro-4-(3-(2-phenylacetyl)thioureido)phenoxy)-7-methoxy-N-methylquinoline-6-carboxamide (compound (1)) was synthesized by the method described in WO 2009/125597.

### Test Example 1

### Antitumor Effect Potentiating Activity for Paclitaxel

A human lung cancer cell line (NCI-H460) was transplanted into the right chest of a 5- to 6-week old male BALB/cA Jcl-nu mouse. After the tumor transplantation, the major axis (mm) and the minor axis (mm) of the tumor were measured to calculate the tumor volume (TV), and then mice were allocated to each treatment group such that the average TV's of each group would be uniform. The day on which this grouping (n=5) was performed was designated as Day 1.

The test solution for a paclitaxel (TAXOL injection, Bristol Myers K.K.) single administration group was prepared such that the administered dose of paclitaxel would be 60 mg/kg/day. Furthermore, the test solution for a compound (1) single administration group was prepared such that the administered dose would be 200 mg/kg/day. The compound (1) was orally administered every day for 14 consecutive days from Day 1, and paclitaxel was administered on Day 1 through the mouse tail vein. In a combined administration group, 200 mg/kg/day of the compound (1) and 60 mg/kg/day of paclitaxel were administered.

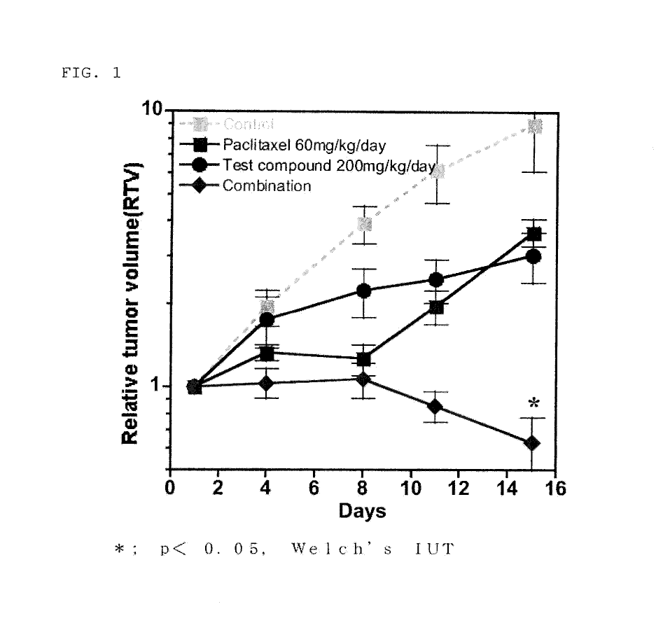

As an index of the antitumor effect, the TV on Day 15 was measured for each medicament treated group, and the relative tumor volume (RTV) for Day 1 and T/C (%) were calculated by the following formulas to evaluate the antitumor effect. For the evaluation and determination of a combined effect, when the mean RTV value of a combined administration group is statistically significantly (Welch's IUT, overall maximum p<0.05) smaller than the mean RTV value of individual single administration groups, it was judged that there was a combined effect. The results are shown in FIG. 1 and Table 1. In the diagram, symbol * represents that a statistically significant difference was observed for the single administration groups.

TV (mm³)=(Major axis×minor axis²)/2

RTV=(TV on Day 15)/(TV on Day 1)

T/C (%)=(Mean RTV value of test solution administered group)/(mean RTV value of control group)×100

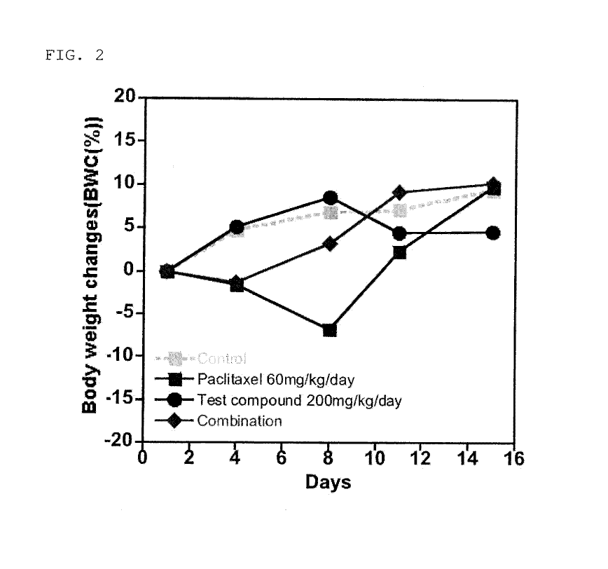

Furthermore, as an index of toxicity, the body weight [BW] was measured over time, and the mean body weight change [BWC (%)] up to Day 15 with respect to Day 1 was calculated by the following formula (n: day of body weight measurement carried out at a rate of two times/week, and the final day of measurement corresponds to Day 15, which is the final evaluation day). The results are shown in FIG. 2.

BWC (%)=[(BW on Day n)−(BW on Day 1)]/(BW on Day 1)×100

TABLE 1

| | | RTV (mean ± | | IUT (Welch) | | |
|---|---|---|---|---|---|---|
| | Dose | standard | | vs | vs | vs Test |
| Group name | (mg/kg/day) | deviation) | T/C (%) | Control | Paclitaxel | compound |
| Control | — | 8.99 ± 2.86 | 100 | — | — | — |
| Paclitaxel | 60 | 3.67 ± 0.42 | 41 | 0.0011 | — | — |
| Test compound | 200 | 3.02 ± 0.63 | 34 | 0.00055 | — | — |
| Combination | 200 + 60 | 0.63 ± 0.15 | 7 | 0.000031 | 0.0000021 | 0.00020 |

### Test Example 2

### Antitumor Effect Potentiating Activity for Gemcitabine Hydrochloride

A human lung cancer cell line (NCI-H441) was transplanted into the right chest of a 5- to 6-week old male BALB/cA Jcl-nu mouse, and the mouse was used in the same manner as described in Test Example 1.

The test solution for a gemcitabine hydrochloride (GEMZAR, manufactured by Eli Lilly and Co.) single administration group was prepared such that the administered dose would be 240 mg/kg/day. Furthermore, the test solution of the compound (1) was prepared such that the administered dose would be 200 mg/kg/day.

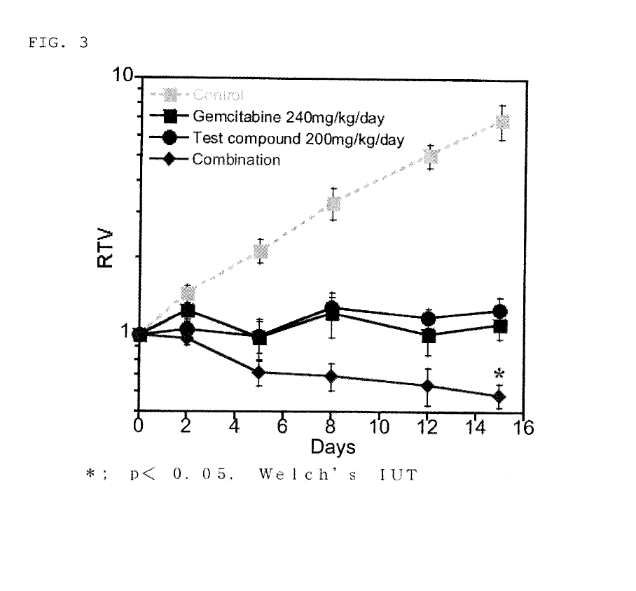

The compound (1) was orally administered every day for 14 consecutive days from Day 1, and gemcitabine hydrochloride was administered on Day 1 and Day 8 through the mouse tail vein. Ina combined administration group, 200 mg/kg/day of the compound (1) and 240 mg/kg/day of gemcitabine hydrochloride were administered, and the effects were evaluated in the same manner as described in Test Example 1. The results are shown in FIG. 3 and Table 2. In the diagram, symbol * represents that a statistically significant difference was observed for the single administration groups.

TABLE 2

| | | RTV (mean ± | | IUT (Welch) | | |
|---|---|---|---|---|---|---|
| | Dose | standard | | vs | vs | vs Test |
| Group name | (mg/kg/day) | deviation) | T/C (%) | Control | Gemcitabine | compound |
| Control | — | 6.92 ± 1.05 | 100 | — | — | — |
| Gemcitabine | 240 | 1.10 ± 0.13 | 16 | 0.00020 | — | — |
| hydrochloride | | | | | | |
| Test compound | 200 | 1.26 ± 0.15 | 18 | 0.00020 | — | — |
| Combination | 200 + 240 | 0.58 ± 0.06 | 8 | 0.00020 | 0.000200 | 0.00020 |

### Test Example 3

### Antitumor Effect Potentiating Activity for TS-1

A human gastric cancer cell line (NUGC-4) was transplanted into the right chest of a 5- to 6-week old male nude rat, and the rat was used in the same manner as described in Test Example 1. The test solution for a TS-1 (TS-1, manufactured by Taiho Pharmaceutical Co., Ltd.) single administration group was prepared such that the amount of administration of tegafur would be 18 mg/kg/day. Furthermore, the test solution of the compound (1) was prepared such that the amount of administration would be 250 mg/kg/day and 50 mg/kg/day.

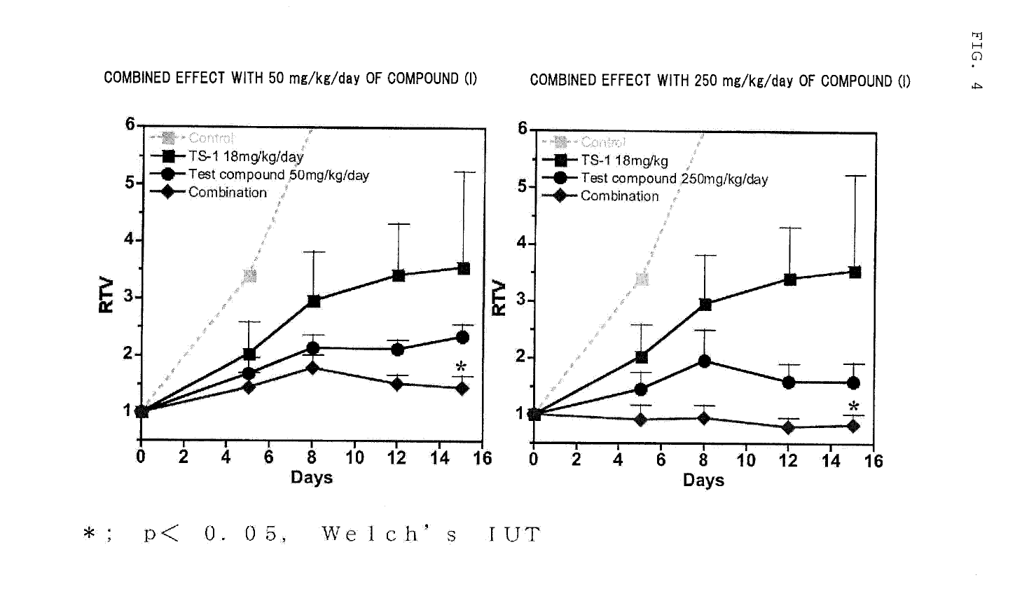

The compound (1) and TS-1 were orally administered every day for 14 consecutive days from Day 1. In a combined administration group, 250 mg/kg/day and 50 mg/kg/day of the compound (1) and 18 mg/kg/day of TS-1 were administered, and the effects were evaluated in the same manner as described in Test Example 1. The results are shown in FIG. 4 and Table 3.

TABLE 3

| | | | | IUT (Welch) | | | |
|---|---|---|---|---|---|---|---|
| | | | | | | vs Test | vs Test |
| | | RTV (mean ± | | | | Compound 1 | compound 1 |
| | Dose | standard | | vs | vs | (50 mg/kg/ | (250 mg/kg/ |
| Group name | (mg/kg/day) | deviation) | T/C (%) | Control | TS-1 | day) | day) |
| Control | — | 16.77 ± 15.01 | 100 | — | — | — | — |
| TS-1 | 18 | 3.55 ± 1.69 | 21 | 0.0028006 | — | — | — |
| Test | 50 | 2.36 ± 0.44 | 14 | 0.001204 | — | — | — |
| compound 1 | | | | | | | |
| Combination | 50 + 18 | 1.45 ± 0.56 | 9 | 0.000630 | 0.0475 | 0.0221 | — |
| Test | 250 | 1.60 ± 0.33 | 10 | 0.000704 | — | — | — |
| compound 1 | | | | | | | |
| Combination | 250 + 18 | 0.83 ± 0.20 | 5 | 0.000406 | 0.0224 | — | 0.0034 |

### Test Example 4

### Antitumor Effect Potentiating Activity for Lapatinib

A human gastric cancer cell line (NCI-N87) was transplanted into the right chest of a 5- to 6-week old male BALB/cA Jcl-nu mouse, and the mouse was used in the same manner as described in Test Example 1. The test solution for a lapatinib (manufactured by LC Laboratories, Inc.) single administration group was prepared such that the amount of administration would be 100 mg/kg/day. Furthermore, the test solution of the compound (1) was prepared such that the amount of administration would be 200 mg/kg/day of the compound (1).

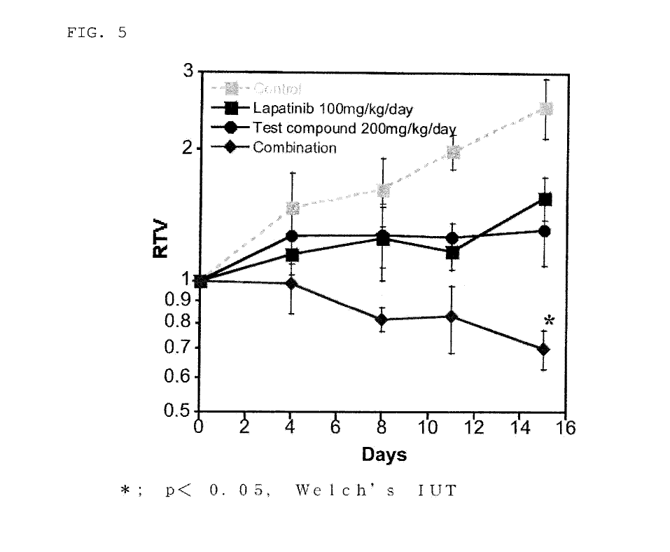

The compound (1) and lapatinib were orally administered every day for 14 consecutive days from Day 1. In a combined administration group, 200 mg/kg/day of the compound (1) and 100 mg/kg/day of lapatinib were administered, and the effects were evaluated in the same manner as described in Test Example 1. The results are shown in FIG. 5 and Table 4. In the diagram, symbol * represents that a statistically significant difference was observed for each single administration group.

TABLE 4

| | | RTV (mean ± | | IUT (Welch) | | |
|---|---|---|---|---|---|---|
| | Dose | standard | | vs | | vs Test |
| Group name | (mg/kg/day) | deviation) | T/C (%) | Control | vs Lapatinib | compound |
| Control | — | 2.52 ± 0.39 | 100 | — | — | — |
| Lapatinib | 100 | 1.56 ± 0.18 | 62 | 0.0010 | — | — |
| Test compound | 200 | 1.31 ± 0.23 | 52 | 0.00020 | — | — |
| Combination | 200 + 150 | 0.70 ± 0.07 | 28 | 0.00010 | 0.000022 | 0.00080 |

### Test Example 5

### Antitumor Effect Potentiating Activity for Irinotecan Hydrochloride

A human colorectal cancer cell line (HT-29) was transplanted into the right chest of a 5- to 6-week old male BALB/cA Jcl-nu mouse, and the mouse was used in the same manner as described in Test Example 1.

The test solution for an irinotecan hydrochloride (CAMPTO injection, manufactured by Yakult Honsha Co., Ltd.) single administration group was prepared such that the amount of administration would be 50 mg/kg/day.

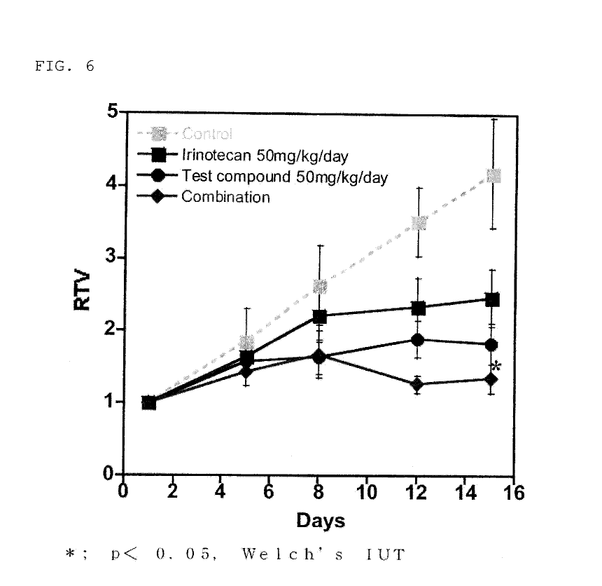

The test solution of the compound (1) was prepared such that the amount of administration would be 50 mg/kg/day of the compound (1). In a combined administration group, 50 mg/kg/day of the compound (1) and 50 mg/kg/day of irinotecan hydrochloride were administered. The compound (1) was orally administered every day for 14 consecutive days from Day 1, and irinotecan hydrochloride was administered on Day 1, Day 5, and Day 9 through the mouse tail vein, and the effects were evaluated in the same manner as described in Test Example 1. The results are shown in FIG. 6 and Table 5. In the diagram, symbol * represents that a statistically significant difference was observed for each single administration group.

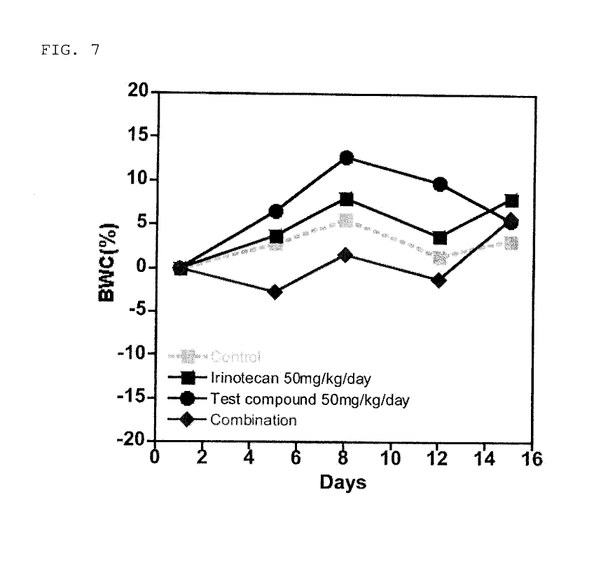

Furthermore, the body weight changes over time, which is an indicator of toxicity, were also evaluated in the same manner as described in Test Example 1. The results are shown in FIG. 7.

TABLE 5

| | | RTV (mean ± | | IUT (Welch) | | |
|---|---|---|---|---|---|---|
| | Dose | standard | | vs | vs Irinotecan | vs Test |
| Group name | (mg/kg/day) | deviation) | T/C (%) | Control | hydrochloride | compound |
| Control | — | 4.19 ± 0.76 | 100 | — | — | — |
| Irinotecan | 50 | 2.47 ± 0.40 | 59 | 0.0079 | — | — |
| hydrochloride | | | | | | |
| Test compound | 50 | 1.83 ± 0.29 | 44 | 0.0079 | — | — |
| Combination | 50 + 50 | 1.35 ± 0.20 | 32 | 0.0079 | 0.0079 | 0.032 |

### Test Example 6

### Antitumor Effect Potentiating Activity for Paclitaxel

A human gastric cancer cell line (NUGC-4) was transplanted into the right chest of a 5- to 6-week old male BALB/cA Jcl-nu mouse, and the mouse was used in the same manner as described in Test Example 1.

The test solution for a paclitaxel (TAXOL injection, manufactured by Bristol Myers K.K.) single administration group was prepared such that the amount of administration of paclitaxel would be 60 mg/kg/day.

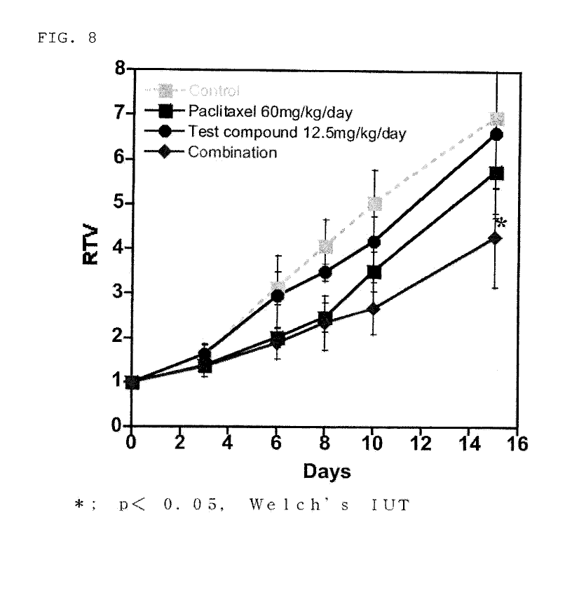

The test solution of the compound (1) was prepared such that the amount of administration would be 12.5 mg/kg/day of the compound (1). In a combined administration group, 12.5 mg/kg/day of the compound (1) and 60 mg/kg/day of paclitaxel were administered. The compound (1) was orally administered every day for 14 consecutive days from Day 1, paclitaxel was administered on Day 1 through the mouse tail vein, and evaluation was made in the same manner as described in Test Example 1. The results are shown in FIG. 8 and Table 6. In the diagram, symbol * represents that a statistically significant difference was observed for each single administration group.

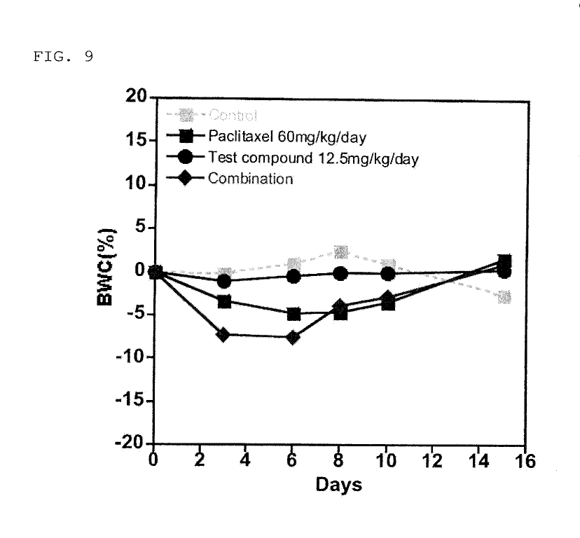

Furthermore, the body weight changes over time, which is an indicator of toxicity, were also evaluated in the same manner as described in Test Example 1. The results are shown in FIG. 9.

TABLE 6

| | | RTV (mean ± | | IUT (Welch) | | |
|---|---|---|---|---|---|---|
| | Dose | standard | | vs | vs | vs Test |
| Group name | (mg/kg/day) | deviation) | T/C (%) | Control | Paclitaxel | compound |
| Control | — | 6.99 ± 1.60 | 100 | — | — | — |
| Paclitaxel | 60 | 5.77 ± 0.96 | 83 | 0.1463 | — | — |
| Test compound | 12.5 | 6.63 ± 1.91 | 95 | 0.7281 | — | — |
| Combination | 12.5 + 60 | 4.27 ± 1.12 | 61 | 0.0078 | 0.032 | 0.031 |

As is obvious from FIGS. 1, 3, 4, 5, 6 and 8, the acylthiourea compound (I) or a pharmaceutically acceptable salt thereof markedly potentiated the antitumor effect of various antitumor agents. The effect was observed from 12.5 mg/kg/day of the acylthiourea compound (I), which is a low dose of the compound (a dose that does not exhibit an antitumor effect, FIG. 8, Table 6), and at 200 mg/kg/day, which is a high dose (maximum effect exhibiting dose) in nude mice, and at 250 mg/kg/day, which is a high dose (maximum effect exhibiting dose) in a nude rat, remarkable shrinkage of tumors was induced by using the acylthiourea compound (I) in combination (Tables 1, 2, 3 and 4). Furthermore, aggravation of body weight reduction was not observed in combined administration (FIGS. 2, 7, and 9). From these, it can be seen that an increase in the efficacy range to at least 16 times or more is secured by combined use of the compound of the present invention and various antitumor agents.

Furthermore, for example, according to comparison of FIG. 1 and FIG. 8, in the case of paclitaxel, even if the same amount of administration is used, differences in the antitumor effect (drug sensitivity) are observed depending on tumors; when paclitaxel was used in combination with the acylthiourea compound (I), potentiation of the effect is observed in both cases. That is, even for a tumor which is less susceptible to paclitaxel which is a drug for combination, when paclitaxel is used in combination with the acylthiourea compound (I), it is expected to potentiate a tumor proliferation suppressing effect of paclitaxel. This implies that combined use with the acylthiourea compound (I) expands the antitumor spectrum of other antitumor agents.

Furthermore, as shown in FIG. 2, in regard to the combined use of the acylthiourea compound (I) and various antitumor agents, since there was no significant difference in body weight reduction as compared with single administration of antitumor agents even in the case of using a high dose of 200 mg/kg/day, it was suggested that toxicity was not potentiated.

That is, it was recognized that the combined administration of the acylthiourea compound (I) or a pharmaceutically acceptable salt thereof and various antitumor agents exhibits a combination effect without exhibiting significant aggravation of toxicity, and expands the therapeutic effect range or the antitumor spectrum. Furthermore, the amount of administration of the acylthiourea compound (I) or a pharmaceutically acceptable salt is desirably set to about 0.005 moles to 50 moles relative to 1 mole of the other antitumor agent.

1-15. (canceled)

16. A composition comprising an acylthiourea compound represented by formula or a pharmaceutically acceptable salt thereof, and another antitumor agent as a single dosage form or as separate dosage forms: 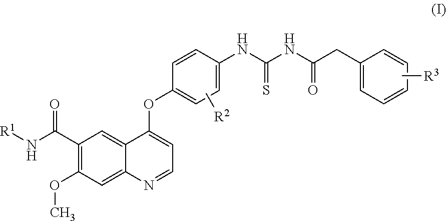
wherein R¹ is an optionally substituted C₁₋₆ alkyl group;
R² is a fluorine atom or a chlorine atom; and
R³ is a hydrogen atom, a fluorine atom, or a chlorine atom; and
wherein the optional substituent on R¹ is selected from the group consisting of a hydroxyl group, a C₃₋₁₀ cycloalkyl group, an optionally substituted C₁₋₆ alkoxy group, an optionally substituted alkylamino group, a C₁₋₆ alkanoylamino group, a C₁₋₆ alkylsulfonyl group, an optionally substituted C₆₋₁₄ aromatic hydrocarbon group, an optionally substituted saturated or unsaturated heterocyclic group, an optionally substituted C₁₋₆ alkylaminocarbonyl group, and an optionally substituted saturated or unsaturated heterocyclic carbonyl group.

17. The composition according to claim 16, wherein the other antitumor agent is selected from the group consisting of paclitaxel, gemcitabine, lapatinib, a tegafur-gimeracil-oteracil potassium combination drug, and irinotecan.

18. The composition according to claim 16,
wherein in formula (I), R¹ is an optionally substituted alkyl group selected from the group consisting of a methyl group, an ethyl group, an n-propyl group, an isopropyl group, an n-butyl group, and a sec-butyl group;
R² is a fluorine atom or a chlorine atom; and
R³ is a hydrogen atom, a fluorine atom, or a chlorine atom;
wherein the optional substituent on R¹ is selected from the group consisting of a hydroxyl group, a cyclohexyl group, a methoxy group, an ethoxy group, an isopropyloxy group, a diethylamino group, an acetylamino group, a methylsulfonyl group, a phenyl group, a pyrrolidinyl group, a morpholino group, a dioxolane group, a tetrahydropyranyl group, a pyridyl group, a triazolyl group, an ethylaminocarbonyl group, a dimethylaminocarbonyl group, a methylbutylaminocarbonyl group, a pyrrolidinylcarbonyl group, and a morpholinocarbonyl group;
and wherein the alkoxy group optionally has a hydroxyl group as a substituent, the heterocyclic group optionally has a methyl group or an oxo group as a substituent, the alkylaminocarbonyl group optionally has a hydroxyl group or a methoxy group as a substituent, the heterocyclic carbonyl group optionally has a fluorine atom or a methyl group which optionally has a hydroxyl group as a substituent.

19. The composition according to claim 16,
wherein R¹ is a hydrogen atom or an optionally substituted alkyl group selected from the group consisting of a methyl group, an ethyl group, an n-propyl group, an isopropyl group, an n-butyl group and a sec-butyl group;
R² is a fluorine atom; and
R³ is a hydrogen atom or a fluorine atom;
and wherein the alkyl group is optionally substituted with a hydroxyl group, a methoxy group or a morpholino group.

20. The composition according to claim 16, wherein the compound of formula (I) is selected from the group consisting of:
(1) 4-(2-fluoro-4-(3-(2-phenylacetyl)thioureido)phenoxy)-7-methoxy-N-methylquinoline-6-carboxamide;
(2) 4-(2-fluoro-4-(3-(2-phenylacetyl)thioureido)phenoxy)-7-methoxy-N-(2-morpholinoethyl)quinoline-6-carboxamide; and
(3) (S)-4-(2-fluoro-4-(3-(2-(4-fluorophenyl)acetyl)thioureido)phenoxy)-N-(1-hydroxybutan-2-yl)-7-methoxyquinoline-6-carboxamide.

21-30. (canceled)

31. A method for potentiating an antitumor effect of another antitumor agent, the method comprising administering the acylthiourea compound of claim 16, or pharmaceutically acceptable salt thereof, and another antitumor agent, to a patient in need thereof.

32. The method according to claim 31,
wherein in formula (I), R¹ is an optionally substituted alkyl group selected from the group consisting of a methyl group, an ethyl group, an n-propyl group, an isopropyl group, an n-butyl group, and a sec-butyl group;
R² is a fluorine atom or a chlorine atom; and
R³ is a hydrogen atom, a fluorine atom, or a chlorine atom;
and wherein the optional substituent on R¹ is selected from the group consisting of has a hydroxyl group, a cyclohexyl group, a methoxy group, an ethoxy group, an isopropyloxy group, a diethylamino group, an acetylamino group, a methylsulfonyl group, a phenyl group, a pyrrolidinyl group, a morpholino group, a dioxolane group, a tetrahydropyranyl group, a pyridyl group, a triazolyl group, an ethylaminocarbonyl group, a dimethylaminocarbonyl group, a methylbutylaminocarbonyl group, a pyrrolidinylcarbonyl group, and a morpholinocarbonyl group;
and wherein the alkoxy group may further have a hydroxyl group as a substituent, the heterocyclic group may further have a methyl group or an oxo group as a substituent, the alkylaminocarbonyl group may further have a hydroxyl group or a methoxy group as a substituent, the heterocyclic carbonyl group may further have a fluorine atom, or a methyl group which may have a hydroxyl group, as a substituent.

33. The method according to claim 31,
wherein R¹ is a hydrogen atom, or an optionally substituted alkyl group selected from the group consisting of a methyl group, an ethyl group, an n-propyl group, an isopropyl group, an n-butyl group and a sec-butyl group;
R² is a fluorine atom; and
R³ is a hydrogen atom or a fluorine atom;
and wherein the alkyl group is optionally substituted with a hydroxyl group, a methoxy group or a morpholino group.

34. The method according to claim 31, wherein the compound of formula (I) is selected from the group consisting of:
(1) 4-(2-fluoro-4-(3-(2-phenylacetyl)thioureido)phenoxy)-7-methoxy-N-methylquinoline-6-carboxamide;
(2) 4-(2-fluoro-4-(3-(2-phenylacetyl)thioureido)phenoxy)-7-methoxy-N-(2-morpholinoethyl)quinoline-6-carboxamide; and
(3) (S)-4-(2-fluoro-4-(3-(2-(4-fluorophenyl)acetyl)thioureido)phenoxy)-N-(1-hydroxybutan-2-yl)-7-methoxyquinoline-6-carboxamide.

35. The method according to claim 31, wherein the other antitumor agent is selected from the group consisting of paclitaxel, gemcitabine, lapatinib, a tegafur-gimeracil-oteracil potassium combination drug, and irinotecan.

36. A method for treating a tumor, the method comprising administering the acylthiourea compound of claim 16, or pharmaceutically acceptable salt thereof, and another antitumor agent, to a patient in need thereof.

37. The method according to claim 36, wherein the other antitumor agent is selected from the group consisting of paclitaxel, gemcitabine, lapatinib, a tegafur-gimeracil-oteracil potassium combination drug, and irinotecan.

38. The method according to claim 36,
wherein in formula (I), R¹ is an optionally substituted alkyl group selected from the group consisting of a methyl group, an ethyl group, an n-propyl group, an isopropyl group, an n-butyl group, and a sec-butyl group;
R² is a fluorine atom or a chlorine atom; and
R³ is a hydrogen atom, a fluorine atom, or a chlorine atom;
and wherein the optional substituent on the alkyl group is selected from the group consisting of a hydroxyl group, a cyclohexyl group, a methoxy group, an ethoxy group, an isopropyloxy group, a diethylamino group, an acetylamino group, a methylsulfonyl group, a phenyl group, a pyrrolidinyl group, a morpholino group, a dioxolane group, a tetrahydropyranyl group, a pyridyl group, a triazolyl group, an ethylaminocarbonyl group, a dimethylaminocarbonyl group, a methylbutylaminocarbonyl group, a pyrrolidinylcarbonyl group, and a morpholinocarbonyl group;
and wherein the alkoxy group optionally has a hydroxyl group as a substituent, the heterocyclic group optionally has a methyl group or an oxo group as a substituent, the alkylaminocarbonyl group optionally has a hydroxyl group or a methoxy group as a substituent, the heterocyclic carbonyl group optionally has a fluorine atom or a methyl group which optionally has a hydroxyl group as a substituent.

39. The method according to claim 36,
wherein R¹ is a hydrogen atom, or an optionally substituted alkyl group selected from the group consisting of a methyl group, an ethyl group, an n-propyl group, an isopropyl group, an n-butyl group and a sec-butyl group;
R² is a fluorine atom; and
R³ is a hydrogen atom or a fluorine atom;
and wherein the alkyl group is optionally substituted with a hydroxyl group, a methoxy group or a morpholino group.

40. The method according to claim 36, wherein the compound of formula (I) is selected from the group consisting of:
(1) 4-(2-fluoro-4-(3-(2-phenylacetyl)thioureido)phenoxy)-7-methoxy-N-methylquinoline-6-carboxamide;
(2) 4-(2-fluoro-4-(3-(2-phenylacetyl)thioureido)phenoxy)-7-methoxy-N-(2-morpholinoethyl)quinoline-6-carboxamide; and
(3) (S)-4-(2-fluoro-4-(3-(2-(4-fluorophenyl)acetyl)thioureido)phenoxy)-N-(1-hydroxybutan-2-yl)-7-methoxyquinoline-6-carboxamide.
